(19) United States
(12) Patent Application Publication
(10) Pub. No.: US 2014/0207128 A1
(43) Pub. Date: Jul. 24, 2014

(54) ABLATION DEVICE

(71) Applicant: TERUMO KABUSHIKI KAISHA, Shibuya-ku (JP)

(72) Inventors: Yoichiro Iwase, Kanagawa (JP); Ichiro Hirahara, Kanagawa (JP); Sayaka Oomori, Kanagawa (JP); Ryota Sugimoto, Kanagawa (JP)

(73) Assignee: TERUMO KABUSHIKI KAISHA, Shibuya-ku (JP)

(21) Appl. No.: 14/226,409

(22) Filed: Mar. 26, 2014

Related U.S. Application Data
(63) Continuation of application No. PCT/JP2012/073777, filed on Sep. 18, 2012.

(30) Foreign Application Priority Data
Sep. 27, 2011 (JP) 2011-211199

(51) Int. Cl. A61B 18/20

(52) U.S. Cl. CPC A61B 18/201
USPC 606/10

(57) ABSTRACT

An ablation device includes a waveguide that guides a pulse laser output from a laser output unit, a laser emitting unit that focuses the pulse laser guided by the waveguide using a lens, emits the pulse laser to a living body tissue, causes multiphoton absorption in a focal position of the pulse laser, and performs ablation on the living body tissue; and a balloon that sets a focus of the pulse laser as the laser emitting unit is positioned at a desired position inside a living body lumen.

### CROSS-REFERENCE TO RELATED APPLICATIONS

This application is a continuation of International Application No. PCT/JP2012/073777 filed on Sep. 18, 2012, and claims priority to Japanese Application No. 2011-211199 filed on Sep. 27, 2011, the entire content of both of which is incorporated herein by reference.

### TECHNICAL FIELD

The present invention generally relates to an ablation device for performing ablation on a desired living body tissue of a living body lumen.

### BACKGROUND DISCUSSION

It has been found that patients with intractable hypertension who find it difficult to improve their high blood pressure even by taking an antihypertensive can expect their blood pressure to be lowered by severing or disrupting the nerve (sympathetic nerve) around the renal artery (living body lumen) to block neurotransmission thereof.

Using an ablation device has been proposed as a medical procedure to sever the nerve of the renal artery. An example is described in Japanese Application Publication No. 2008-515544 which discloses a device (renal neuromodulation device) for severing the nerve around the renal artery. The device is configured with a helical electrode disposed around an expandable balloon and the helical electrode is brought into contact with a blood vessel wall (inner wall) of the renal artery for energizing. Similarly, Japanese Application Publication No. 2010-509032 discloses a configuration where an electrode is disposed around a positioning member such as a balloon or the like, where the balloon is expanded and the electrode is brought into contact with the inner wall of the renal artery for energizing.

The devices disclosed in Japanese Application Publication No. 2008-515544 and Japanese Application Publication No. 2010-509032 are configured to bring the electrode into contact with the inner wall of the renal artery (living body lumen) for energizing, thereby performing ablation on all living body tissues including the inner wall of the living body lumen. The term “ablation” used herein refers to general therapies including removal, excision, cauterization, fusion, perspiration, disruption and injury of the living body tissues. Particularly when the laser is used (also referred to as laser ablation), the term “ablation” includes a process for performing the above-described therapies on the living body tissue by converting the energy of the laser into electronic, thermal, photochemical and mechanical energy.

### SUMMARY

The devices disclosed in Japanese Application Publication No. 2008-515544 and Japanese Application Publication No. 2010-509032 supply electric current to an inner wall of the renal artery when severing the nerve of the renal artery. Accordingly, there is a possibility that the electric current may flow to other living body tissues and cause an adverse effects on a living body (for example, unexpected activity of cells, spasm of the blood vessel, nerve stimulation or the like), thereby increasing issues to the living body in surgery.

In addition, as described above, the renal artery is subjected to cauterization (ablation) by means of energizing. Thus, energy is also transmitted to the nerves other than an ablation target and causes the living body tissues to be cauterized. In this case, thrombosis or arterial dissection (aneurysm) is likely to occur near a cauterizing point of the renal artery.

Furthermore, when severing the nerve of the renal artery, it is necessary to sever all the nerves in a circumferential direction of the renal artery. In this case, if the cauterization is electrically performed, an injury to the intima is involved. Accordingly, it is not possible to cauterize all around the nerve at once. Therefore, the renal artery is not coaxially cauterized and a plurality of cauterizing points (segments) is set in an axial direction. Then, discontinuous boundary treatment is carried out for severing the nerve of the renal artery so that the segments are not overlapped with each other. For this reason, a medical procedure requires a great deal of time (for example, approximately 40 minutes), thereby imposing severe burden on a patient.

The medical ablation device disclosed here can significantly reduce the burden on the patient by suppressing injury to a surface of the living body lumen and more accurately performing the ablation on a desired living body tissue.

In accordance with one aspect, an ablation device includes a catheter that can be inserted into a living body lumen, a waveguide disposed along the catheter to guide a pulse laser output from a laser output unit; and a laser emitting unit that has a laser focusing unit which focuses the pulse laser guided by the waveguide on a desired living body tissue, and that emits the pulse laser so as to perform ablation on the living body tissue by causing multiphoton absorption to occur in a focal position of the pulse laser.

The pulse laser guided to the laser emitting unit via the waveguide is focused and emitted to the desired living body tissue by the laser emitting unit, and the multiphoton absorption is caused to occur in or at the focal position. In this manner, it is possible to rather easily perform the ablation on the living body tissue located at the focal position. In this case, the pulse laser can be transmitted to the focal position on a wavelength which is less likely to be absorbed in the living body tissue, and at the focal position, the pulse laser can be changed to have a wavelength which is likely to be absorbed in the living body tissue. Accordingly, without imposing damage to the surface of the living body lumen, it is possible to perform the ablation on a target region (focal position) inside the living body tissue. As a result, it is possible to demonstrate a procedure in a relatively short time, without causing thrombosis, aneurysm or the like in the living body lumen. Therefore, it is possible to greatly reduce the burden on a patient.

In addition, since optical energy generated by emission of the pulse laser is used, it is possible to significantly reduce influence on a living body as compared to a case of energizing the living body.

Positioning means for setting a focus of the pulse laser may also be provided, as the laser emitting unit is positioned at a desired position inside the living body lumen.

The laser emitting unit is positioned at the desired position inside the living body lumen by the positioning means. Therefore, it is possible to relatively exactly and simply set the focus of the pulse laser. As a result, the laser emitting unit can accurately focus the pulse laser on a desired living body tissue to perform the ablation.

According to one aspect, it is preferable that the positioning means be a balloon which is disposed on a side peripheral surface of the catheter and which is radially expandable inside the living body lumen.

When the balloon is used as the positioning means and the balloon is delivered to the desired position inside the living body lumen, the balloon is delivered in a contracted state. When the balloon reaches the desired position, if the balloon is expanded, the laser emitting unit can be relatively easily positioned and fixed thereto inside the living body lumen.

In this case, by leaving the balloon in an expanded state inside the living body lumen, a central axis of the catheter may be allowed to coincide with a central axis of the living body lumen.

In this manner, the expansion of the balloon allows the central axis of the catheter to coincide with (be coaxial with) the central axis of the living body lumen. Accordingly, it is possible to radially equalize the distance from the central axis of the catheter to the living body tissue inside the living body lumen. That is, it is possible to easily center the central axis of the catheter on the central axis of the living body lumen. Therefore, without changing a focal length of the pulse laser, the ablation can be performed in the circumferential direction of the living body lumen.

In addition, the balloon may be disposed at a position axially overlapping with the laser focusing unit in an axial direction of the catheter.

In this manner, since the balloon is disposed at the axially overlapping position with the laser focusing unit, it is possible to suppress positional deviation or shaking of the laser emitting unit in a positioning point of the laser emitting unit with respect to the living body lumen. In addition, in a direction where the laser focusing unit emits the pulse laser, it is possible to suppress intervention of blood which tends to scatter the pulse laser. Accordingly, it is possible to more excellently perform the ablation by using the pulse laser.

The balloon may also be disposed at a neighboring position of the laser focusing unit in the axial direction of the catheter.

Even when the balloon is disposed at the neighboring position of the laser focusing unit, it is possible to suppress the positional deviation or the shaking of the laser emitting unit. In addition, it is possible to allow the central axis of the living body lumen to coincide with (to be centered on) the central axis of the catheter. In this case, it is possible to block the blood flowing inside the living body lumen by expanding the balloon. Accordingly, it is possible to reduce a possibility that the pulse laser emitted from the laser emitting unit may be influenced by the flowing of the blood.

Furthermore, the balloon in the inflated state may be configured as the laser focusing unit.

In this manner, the balloon configures the laser focusing unit. Accordingly, the balloon which is expanded for proper positioning enables the pulse laser to be focused on a target region of the desired living body tissue. In addition, since it is not necessary to dispose a lens for focusing the pulse laser in the laser emitting unit, it is possible to reduce the manufacturing cost for the ablation device.

Here, it is preferable that the laser focusing unit be disposed to face the living body lumen from a side peripheral surface of the catheter and the laser emitting unit have a reflection unit which guides the pulse laser guided by the waveguide to the laser focusing unit.

Because the laser focusing unit faces the living body lumen from the side peripheral surface of the catheter, it is possible to emit the pulse laser from the side peripheral surface of the catheter. Accordingly, when inserting the catheter, it is possible to relatively easily perform the ablation on the living body lumen located on a delivery route of the catheter.

It is preferable that the laser emitting unit be rotatable inside the catheter in a circumferential direction of the catheter.

Since the laser emitting unit is rotatable inside the catheter in the circumferential direction of the catheter, it is possible to perform the ablation on all the living body tissues in the circumferential direction inside the living body lumen. Therefore, for example, in procedure for performing the ablation on the nerve of the renal artery, it is possible to perform the ablation on all the nerves internally passing through the renal artery in the positioning point of the ablation device. Accordingly, it is possible to significantly improve work efficiency.

It is also preferable to employ a configuration where the laser output unit is provided in a base end side of the catheter and the laser output unit emits a pulse laser whose pulse width is shorter than that of one nanosecond.

By emitting the pulse laser whose pulse width is shorter than that of one nanosecond, it is possible to easily cause multiphoton absorption at the focal position.

Furthermore, the waveguide may be configured to have a hollow shape surrounded by a waveguide tube through which the pulse laser can be transmitted.

In this manner, the waveguide is formed to have the waveguide tube having a hollow structure. Accordingly, even when the pulse laser has a short pulse width, it is possible to suppress a transmission loss and to guide the pulse laser to the laser emitting unit. Therefore, it is possible to easily emit the pulse laser having predetermined laser intensity from the laser emitting unit.

According to the present invention, the ablation is performed precisely on the desired living body tissue while suppressing injury to the surface of the living body lumen. Therefore, it is possible to greatly reduce the burden on the patient.

According to another aspect, a medical device to ablate living body tissue includes a catheter sized to be inserted into a living body lumen, a pulse laser output unit which outputs ultra-short pulse laser, a waveguide in the catheter, with the waveguide being connectable to the pulse laser output unit to guide the ultra-short pulse laser output by the pulse laser output unit, and a focusing lens which focuses the ultra-short pulse laser PL guided along the waveguide on a focal position located on the living body tissue, and emits the pulse laser to ablate the living body tissue by causing multiphoton absorption at the focal position.

In accordance with another aspect, a method of ablating living body tissue of a living body comprises positioning a waveguide in a living body lumen of the living body, guiding a pulse laser along the waveguide, and focusing the pulse laser guided along the waveguide at a focal position on the living body tissue and emitting the pulse laser toward the living body tissue to perform ablation on the living body tissue by causing multiphoton absorption at the focal position.

### BRIEF DESCRIPTION OF DRAWINGS

FIG. 1A is a schematic view illustrating excitation of a photon in a normal laser.

FIG. 1B is a schematic view illustrating excitation of photons when a pulse laser causes two-photon absorption.

FIG. 1C is a schematic view illustrating a state where the normal laser is emitted to a living body tissue.

FIG. 1D is a schematic view illustrating a state where the pulse laser is emitted to the living body tissue.

FIG. 2 is a schematic explanatory view illustrating an overall configuration of a first embodiment of an ablation device representing one example of the ablation device disclosed here.

FIG. 3A is a somewhat schematic side cross-sectional view illustrating a balloon before being expanded in the ablation device of FIG. 2 in a state where the ablation device is inserted into the renal artery.

FIG. 3B is a somewhat schematic side cross-sectional view illustrating the balloon after being expanded in the ablation device of FIG. 2 in the state where the ablation device is inserted into the renal artery.

FIG. 4 is a somewhat schematic side cross-sectional view illustrating a state where ablation is performed on a target region of a living body tissue by using the ablation device of FIG. 2.

FIG. 5 is a somewhat schematic side cross-sectional view illustrating a modified example of the ablation device according to the first embodiment.

FIG. 6A is a somewhat schematic explanatory view illustrating a balloon before being expanded in an ablation device according to a second embodiment representing another example of the ablation device disclosed here.

FIG. 6B is a somewhat schematic explanatory view illustrating a balloon after being expanded in the ablation device according to the second embodiment.

### DETAILED DESCRIPTION

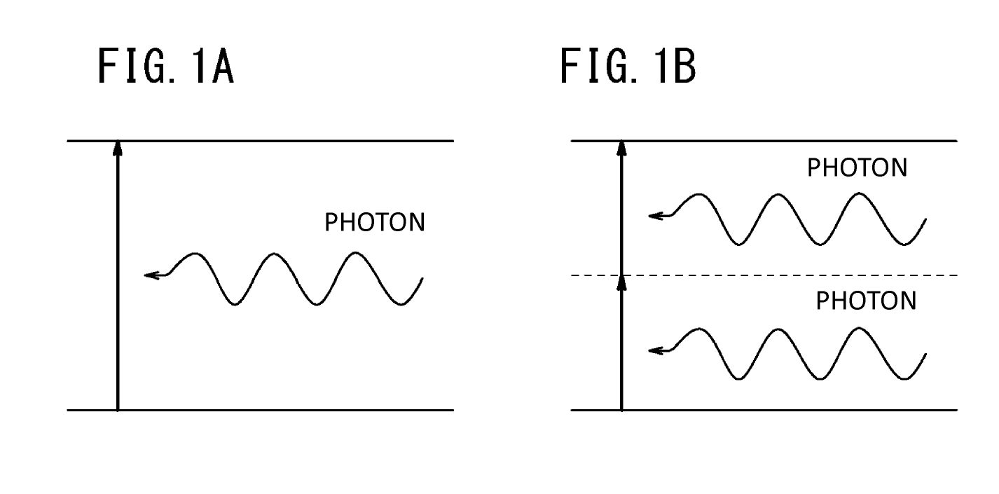

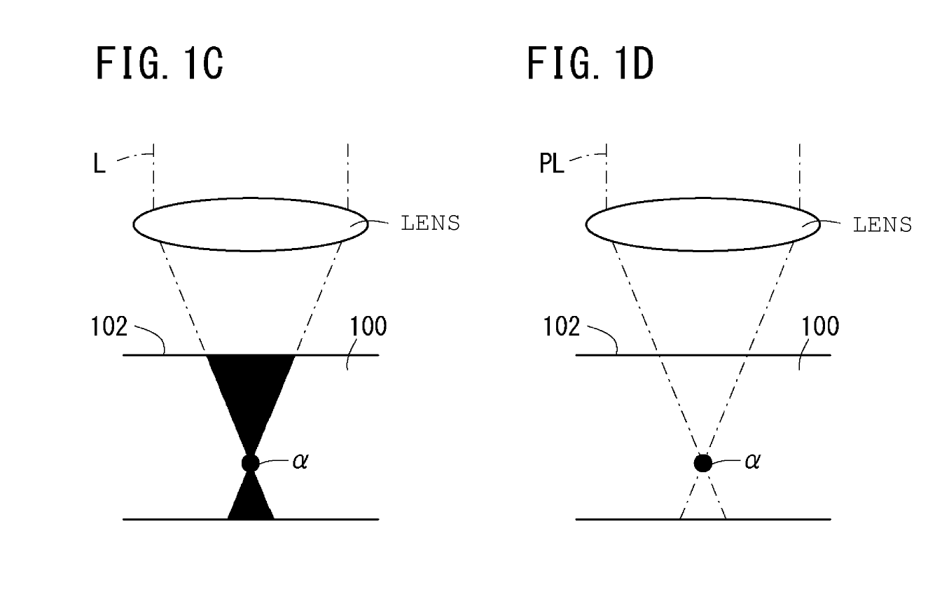

An ablation device (hereinafter, simply referred to as a device) 10 disclosed here is configured to focus a pulse laser on a desired living body tissue, and cause multiphoton absorption (two-photon absorption) in a focal position of the pulse laser, thereby performing ablation on the living body tissue. First, a principle of two-photon absorption will be briefly described with reference to FIGS. 1A-1D.

FIG. 1 A illustrates excitation of a photon in a normal laser L, FIG. 1B illustrates excitation of photons when a pulse laser PL causes two-photon absorption, FIG. 1C illustrates a state where the normal laser L is emitted toward living body tissue and FIG. 1D illustrates a state where the pulse laser PL is emitted to the living body tissue.

A method has been known in which for the purpose of treatment or examination of a living body, a laser beam (laser L) is emitted to a living body tissue 100 to perform ablation on the living body tissue 100 (for example, removal, cauterization, perspiration, photochemical physical reaction or the like). In this case, as illustrated in FIG. 1A, the laser L has energy in which one photon causes an excited state of the living body tissue 100, and as illustrated in FIG. 1C, the ablation is performed from a surface of the living body tissue 100.

In contrast, the device disclosed here is configured so that a pulse laser PL having a sufficiently short pulse width is emitted to (at) the living body tissue 100 to cause two-photon absorption. Two-photon absorption is a phenomenon where two-photons are simultaneously absorbed at a predetermined point (focal position α) so as to cause a state of electrons or atoms to be excited and to be transited to a high energy level (refer to FIG. 1B). In the pulse laser PL whose pulse width is shorter than that of one nanosecond (ultra-short pulse laser, also referred to as a femtosecond laser particularly when the pulse width is that of femtosecond laser), it is possible to relatively easily cause two-photon absorption. That is, it has been known that in a region having a small absorption cross-sectional area, the probability of the two-photon absorption process occurring is proportional to a square of light intensity (photon density)(third order nonlinear effect). Accordingly, if a pulse of high intensity (giant pulse) is emitted, it is possible to greatly increase the probability of the two-photon absorption process occurring even when using a laser having the samaverage intensity.

By causing two-photon absorption to occur, it is possible to obtain square-law characteristics in which fluorescence is generated in proportion to the square of excitation light intensity and wavelength characteristics in which the wavelength of excitation light doubles that of a case of single photon excitation. The ablation device and method here utilize both of these characteristics, thereby performing the ablation on the desired living body tissue 100. That is, as illustrated in FIG. 1D, the pulse laser PL is emitted up to the focal position α of the pulse laser PL by using the wavelength which can be easily transmitted through (can hardly be absorbed in) the living body tissue 100. Then, two-photon absorption is caused to occur at the focal position α so as to obtain the wavelength which is likely to be absorbed in the living body tissue 100 (doubled wavelength). In this manner, for example, without imposing damage to a surface of a living body lumen 102, it is possible to perform the ablation on the desired living body tissue 100 inside the living body lumen 102. Furthermore, in a target region (focal position α) of the living body tissue 100, by utilizing the square-law characteristics, it is possible to localize heat generation caused by optical, physical and chemical reactions or light absorption. Therefore, it is possible to efficiently perform the ablation on the target region (focal position α) of the desired living body tissue 100.

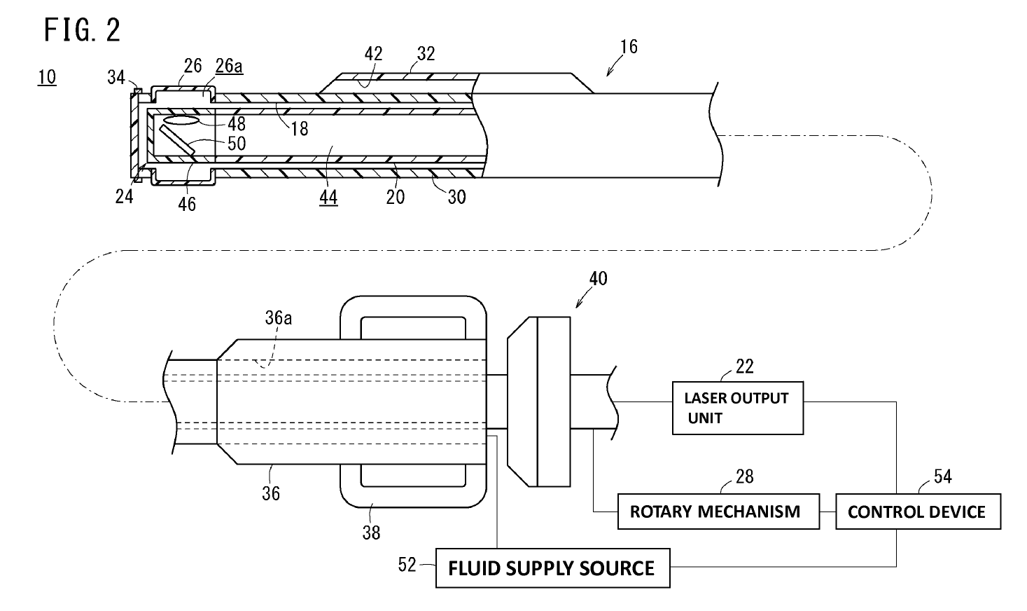

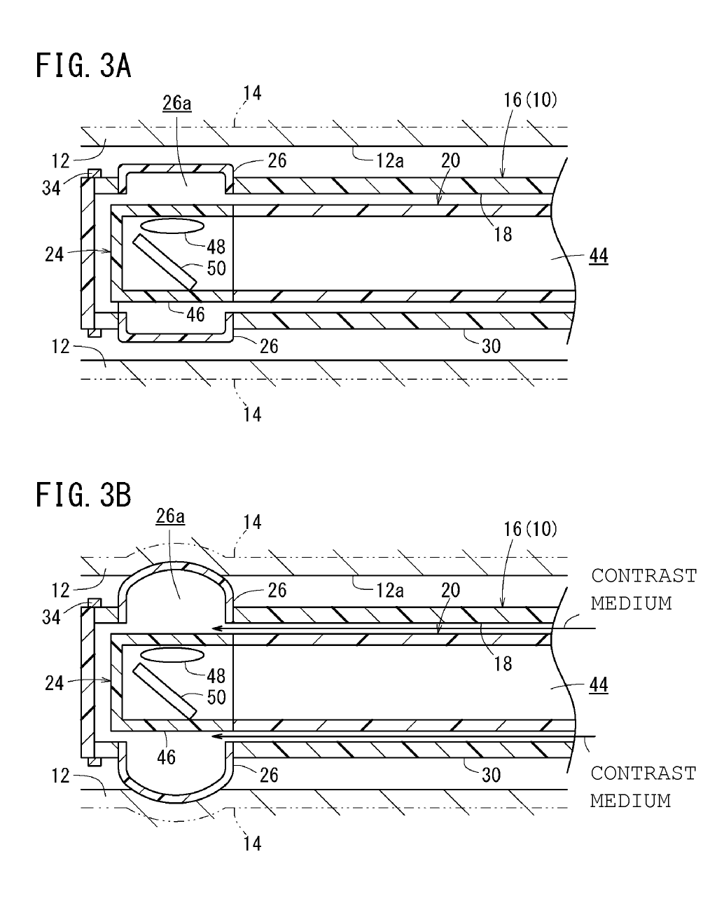

An ablation device 10 disclosed here which utilizes the two-photon absorption principle discussed above will be described with reference to FIGS. 2, 3A and 3B. FIG. 2 illustrates an overall configuration of the ablation device 10, while FIG. 3A illustrates a balloon 26 used in the ablation device 10 of FIG. 2 before being expanded in a state in which the ablation device 10 is positioned in a renal artery 12 and FIG. 3B illustrates the balloon 26 after being expanded in a state in which the ablation device 10 is positioned in a renal artery 12.

The device 10 according to the first embodiment is a medical device for performing ablation on a nerve 14 of a living body lumen so as to perform severance on the nerve 14. In the embodiment disclosed by way of example, the device 10 is a medical device for performing ablation on a sympathetic nerve of the renal artery 12 to perform severance on the nerve 14. The device 10 is disposed in a catheter 16 which can be inserted into the renal artery 12. The catheter 16 is inserted through a predetermined region (for example, femoral artery), thereby delivering the device 10 to a desired position (treatment point) of the renal artery 12. Then, the device 10 has a function for blocking the nerve 14 of the renal artery 12 by emitting the pulse laser PL to the treatment point.

In the following description, the device 10 performs ablation on the nerve 14 of the renal artery 12, but the device 10 is not limited in this regard. For example, the device 10 may be configured to serve as a medical device for performing the ablation on blood vessels other than the renal artery 12, alternatively, on the living body tissue of a living body lumen such as the biliary, trachea, esophagus, urethra, vagina, uterus and the like.

As illustrated in FIG. 2, the device 10 includes the catheter 16, a waveguide tube 20 which is disposed in a lumen 18 (inner cavity) of the catheter 16 and guides the pulse laser PL, a laser output unit 22 which outputs the pulse laser PL to the waveguide tube 20, a laser emitting unit 24 which emits the pulse laser PL outward from the catheter 16, a balloon (positioning means) 26 for positioning the catheter 16, and a rotary mechanism 28 for performing a rotary operation of the laser emitting unit 24.

Hereinafter, a right side (hub 36 side) of the catheter 16 in FIG. 2 is referred to as a “base end (rear end, rear)” side, and a left side (laser output unit 22 side) of the catheter 16 is referred to as a “distal end (front)” side so as to describe the device 10.

With regard to the catheter 16, a known medical device can be used. In this case, as illustrated in FIG. 2, the device 10 can be provided with a so-called rapid exchange type of the catheter 16 which includes a shaft body 30 which internally has the lumen 18 and extends from the base end to the distal end, and a guide portion 32 to be connected to a side peripheral surface near a distal end portion of the shaft body 30.

The shaft body 30 configures an outer appearance of the catheter 16. In view of operability thereof (shape, friction resistance, flexibility, durability and the like), the shaft body 30 is formed so that the catheter 16 can be easily delivered inside the blood vessels (living body lumen). Configuring materials of the shaft body 30 are not particularly limited, but for example, preferably employs polyolefin such as polypropylene, polyethylene and ethylene-vinyl acetate copolymer, polyester such as polyamide, polyethylene terephthalate and polybutylene terephthalate, a fluorine-based resin such as polyurethane, polyvinyl chloride, polystyrene-based resin and ethylene/tetrafluoro ethylene copolymer, various flexible resins such as polyimide, high polymer materials of various elastomers or mixtures thereof such as polyamide elastomers, polyester elastomers, polyurethane elastomers, polystyrene elastomers, fluorinated elastomers, silicone rubber and latex rubber, and alternatively a multilayer tube of the high polymer materials composed of two or more materials described above.

If the catheter 16 is used in the context of the renal artery 12, the shaft body 30 can employ an outer shape of approximately 300 mm to 1,500 mm in total length and approximately 1 mm to 20 mm in outer diameter, for example. It is more preferably to set the total length to be approximately 500 mm to 1,000 mm and the outer diameter to be approximately 3 mm to 10 mm.

A waveguide tube 20 is coaxially inserted into the lumen 18 of the shaft body 30 (catheter 16). That is, if the catheter 16 is inserted into or positioned in the blood vessel (living body lumen), the device 10 can integrally deliver (move forward and rearward) the shaft body 30 and the waveguide tube 20. Dimensions of the lumen 18 depend on the outer diameter of the catheter 16. When the catheter 16 is inserted into the renal artery 12, the dimensions employed can include a diameter of approximately 1 mm to 20 mm, for example. It is more preferable to set the diameter to be approximately 3 mm to 10 mm.

A marker 34 for X-ray contrast is disposed on a distal end peripheral (outer) surface of the shaft body 30. The marker 34 enables a user to visibly check or identify the position of the distal end portion of the shaft body 30 during X-ray illumination. In this case, it is preferable to form the marker 34 by using X-ray impermeable (radiopaque) materials (radiopaque materials such as gold, platinum, tungsten and the like, for example). A position for forming the marker 34 is not necessarily limited to the distal end portion of the shaft body 30, but it is preferable to set the marker 34 to be located at a neighboring position of the balloon 26 serving as the positioning means. By forming the marker 34 at the neighboring position of (i.e., adjoining) the balloon 26, the user can recognize the positioning point of the device 10 in the renal artery 12 to advantageously perform the ablation.

The hub 36, functioning as a gripping portion when operating the catheter 16, is connected to the base end portion of the shaft body 30. A handle 38 is disposed on the outer peripheral surface of the hub 36 to facilitate a user's operation. In addition, an insertion path 36a is axially formed inside the hub 36, and the base end side of the shaft body 30 is inserted and fixed to the insertion path 36a. If the user operates the hub 36 of the base end side, the device 10 performs a forward/backward movement and rotary operation of the catheter 16 on the living body lumen. The inserted waveguide tube 20 internally passing through the lumen 18 of the shaft body 30 comes out (extends out) from the base end side of the insertion path 36a.

In addition the guide portion 32 of the catheter 16 internally has a guide wire lumen 42 through which a guide wire is inserted. The guide wire is inserted through the guide wire lumen 42 before the catheter 16 is delivered, and enters the renal artery 12. Then, the catheter 16 is guided by the guide portion 32 into which the guide wire is inserted, thereby being delivered to the desired position (treatment point) of the renal artery 12.

The waveguide tube 20 is a tubular body extending into the lumen 18 of the catheter 16 (shaft body 30), and internally has a hollow waveguide 44. That is, the waveguide tube 20 is configured to include a hollow fiber (hollow structure) having air (gas) as a core (waveguide 44). In this manner, since the hollow fiber is provided, it is possible to eliminate reflection losses in a fiber end of a giant pulse laser. Therefore, it is possible to obtain high transmission efficiency.

In the waveguide tube 20, the laser output unit 22 (refer to FIG. 2) is connected to the base end side and the laser emitting unit 24 is connected to the distal end side. The pulse laser PL output from the laser output unit 22 is guided to the laser emitting unit 24 through the waveguide 44 of the waveguide tube 20.

The material of the waveguide tube 20 depends on the wavelength of the pulse laser PL transmitting through the waveguide 44. However, it is preferable to use a material (for example, metal such as copper) whose reflective index n is smaller than 1 in the transmission wavelength. This allows the pulse laser PL to be transmitted while being totally reflected on a tube wall (inner wall) of the waveguide tube 20.

The dimensions of the waveguide 44 depend on the diameter of the lumen 18. When the catheter 16 is inserted into the renal artery 12, the dimensions can employ a diameter of approximately 1 mm to 20 mm, for example. It is more preferable to set the diameter to be approximately 3 mm to 10 mm.

The waveguide tube 20 is not limited to the above-described configuration. For example, a known fiber structure using quartz glass as the core may be utilized. In addition, a configuration may be adapted in which the inner wall of the waveguide tube 20 (hollow fiber) is coated with a dielectric layer to increase reflectivity of the pulse laser PL and to decrease transmission losses. Furthermore, the first embodiment adapts the configuration in which the waveguide tube 20 is inserted (extended) into or positioned in the lumen 18 of the catheter 16. However, the lumen 18 itself can be configured to serve as the waveguide 44. In this case, it is possible to transmit the pulse laser PL by forming the inner wall of the catheter 16 using the same material as that of the waveguide tube 20 described above.

The laser output unit 22 to be connected to the base end side of the waveguide tube 20 generates the pulse laser PL whose laser emitting time (pulse width) is short, that is, a so-called ultra-short pulse laser. For example, the laser output unit 22 is configured to output the pulse laser PL having the pulse width which is shorter than that of one nanosecond, more preferably the pulse laser PL having the pulse width of one femtosecond. In this manner, it is possible to relatively easily cause non-linear multiphoton absorption (two-photon absorption) using the pulse laser PL which is to be described later.

More specifically, the laser output unit 22 employs titanium (Ti)-doped sapphire, so-called titanium sapphire (not illustrated), as a laser medium of a laser source. The laser output unit 22 is configured to output a mode-locked laser having a wavelength region in the near-infrared. The titanium sapphire can obtain the wavelength of 800 nm as a basic wavelength of the pulse laser PL. It has been known that the wavelength of 800 nm falls within a region of the wavelength which is less likely to be absorbed in the living body. Accordingly, there is an advantage in that the wavelength can be rather easily transmitted through blood vessel wall of the renal artery 12 (refer to FIG. 3A).

In this case, for example, the titanium sapphire is arranged between a pair of laser resonators. In this manner, the excitation light (for example, an argon laser, Nd:YVO₄ laser and the like) incident from one laser resonator has or exhibits a high frequency by the laser resonator, and can be emitted from the other laser resonator as the pulse laser PL. The laser output unit 22 of the first embodiment is configured to output the pulse laser (giant pulse) in which the wavelength is 800 nm, the pulse width is 100 fs (10⁻¹³ seconds), the repetition frequency is 50 MHz and the output is 0.5 mW to 1 mW.

The laser emitting unit 24 has a cap 46 which extends to or is positioned at the distal end portion of the waveguide tube 20. Inside the cap 46, there are provided a focusing lens or (laser focusing unit) 48 which focuses the pulse laser PL on the predetermined focal position a, and a mirror 50 which reflects the pulse laser PL guided by the waveguide 44. The cap 46 can be moved integrally with the waveguide tube 20, and accordingly, the lens 48 and the mirror 50 are also configured to be integrally movable. The cap 46 may be formed of a material which is the same as that of the waveguide tube 20, or a material having a light blocking effect. However, it is preferable to form a peripheral surface portion (upper portion side of the cap 46 illustrated in FIG. 2) corresponding to the installation point of the lens 48, by using a material through which the pulse laser PL can be relatively easily transmitted (for example, glass, plastic or the like).

The lens 48 of the laser emitting unit 24 is a biconvex lens made of a material through which the pulse laser PL having the above-described output condition can be transmitted and which focuses the pulse laser PL (for example, glass, plastic or the like). The lens 48 is arranged on an inner peripheral surface of the cap 46 so that an optical axis of the lens faces a side peripheral surface of the catheter 16 (that is, so as to be orthogonal to the axis of the waveguide 44). In addition, in the lens 48, the focal position α (focal length D) of the pulse laser PL incident on the lens 48 is set based on an expansion amount of the balloon 26 and a distance from an intima surface 12a of the renal artery 12 to the nerve 14. Setting of the focal length D will be described later.

The mirror 50 is internally attached or fixed to the cap 46 in a state of being tilted by a predetermined angle (for example, 45°). The mirror 50 is formed in a shape which covers the entire surface of the waveguide 44 (entire surface in a travelling direction of the pulse laser) when seen in a front cross-sectional view of the catheter 16. Therefore, the pulse laser transmitted through the waveguide 44 is reflected to the lens 48 in a state of having suppressed losses.

The balloon 26 is means for preventing movement (fixing) of the catheter 16 relative to the renal artery 12 (living body lumen), and is disposed in a circumferential direction of the side peripheral surface of the catheter 16. In addition, the balloon 26 also serves as the positioning means which has a centering function for positioning the central axis of the distal end portion of the catheter 16 to coincide with the central axis of the renal artery 12. The balloon 26 has a chamber 26a in which a volume (internal pressure) is changed or increased by fluid supply, and the chamber 26a is connected to the lumen 18 of the shaft body 30. A fluid supply source 52 which supplies a fluid to the interior of the balloon to expand the balloon 26 is connected to the base end side of the lumen 18.

The fluid for expanding the balloon 26 can include a contrast medium which does not absorb the pulse laser PL, a fluid such as saline, or a gas including air. In the device 10 according to the first embodiment, a configuration for supplying the contrast medium to the balloon 26 will be described.

When the catheter 16 is delivered, the balloon 26 is in a contracted state where the contrast medium is not supplied to the interior of the balloon. When the catheter 16 reaches a desired position, the balloon 26 is changed to be in an expanded state by the contrast medium being supplied from the fluid supply source 52. This enables the catheter 16 to be positioned and fixed (positionally fixed) inside the renal artery 12. In the expanded state of the balloon 26, the central axis of the catheter 16 overlapped with the balloon 26 coincides with the central axis of the renal artery 12.

Here, the balloon 26 according to the first embodiment is disposed at a position overlapping with the installation position of the laser emitting unit 24 (lens 48) in the axial direction of the catheter 16. That is, the balloon 26 and the lens 48 are positioned in axially overlapping relation to one another. Therefore, in a state where the catheter 16 is positioned, the lens 48 opposes the expanded balloon 26. Thus, the device 10 can regulate a change in the focal position α of the lens 48 by using the balloon 26. In this manner, the device 10 can rather easily focus the pulse laser PL emitted from the lens 48 on the nerve 14 located within a range of 0.1 mm to 2 mm from the intima of the renal artery 12.

The material of the balloon 26 requires proper flexibility, and requires enough strength to be reliably positioned in the renal artery 12. Furthermore, it is preferable to use a material which enables the pulse laser PL to be excellently transmitted through the balloon 26. In this case, for example, the balloon 26 can employ polyolefin, polyolefin elastomer, polyester, polyester elastomer, polyamide, polyamide elastomer, polyurethane, polyurethane elastomer, polyethylene terephthalate, styrene-olefin rubber, or the like. A blended material in which two or more types of resins are mixed or a material having a multilayer structure obtained by stacking (laminating) two or more types of resins may be used. In addition, the balloon 26 can be configured so that in the expanded state, the outer diameter is, for example, approximately 1 mm to 20 mm in the case of the catheter 16 being positioned in the renal artery 12. It is preferable to set the outer diameter to be approximately 3 mm to 10 mm.

The rotary mechanism 28 of the device 10 is connected to the waveguide tube 20 extending from the base end side of the hub 36, and has a function for circumferentially rotating the waveguide tube 20 inside the catheter 16 in a state of positioning and fixing the catheter 16. In this case, in a state of controlling the waveguide tube 20 at a desired rotational speed by using a rotary drive source such as a servo motor, the rotary mechanism 28 transmits a rotary drive force to the waveguide tube 20 and rotates the waveguide tube 20 at a constant speed. The laser emitting unit 24 emits the pulse laser PL and is rotated in the circumferential direction of the catheter 16, thereby enabling the pulse laser PL to be emitted in the circumferential direction of the renal artery 12. The rotary mechanism 28, without being limited to the rotary drive source such as the servo motor, may employ various mechanisms as a matter of course.

In addition, the waveguide tube 20 extending out from the base end side of the hub 36 has an operation unit 40 whose diameter increases in the radial direction. A user manually performs rotary operation on the operation unit 40, thereby enabling the waveguide tube 20 to be rotated. In this manner, the manual operation of the waveguide tube 20 enables the user to perform delicate positioning adjustment in the rotating direction of the waveguide tube 20 (laser emitting unit 24).

The laser output unit 22, the rotary mechanism 28 and the fluid supply source 52 are connected to and controlled by a control device 54. The user operates an on-off switch to work or operate the laser output unit 22, the rotary mechanism 8 and the fluid supply source 52 respectively during the procedure.

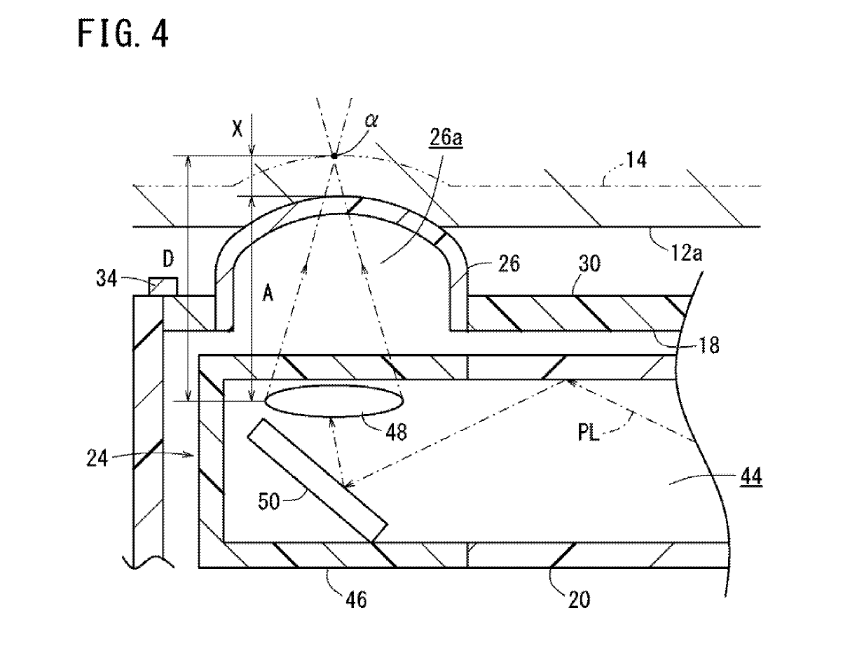

The device 10 according to the first embodiment is configured as described above. Subsequently, a method of performing the ablation on the nerve 14 of the renal artery 12 by using the device 10 will be described with reference to FIGS. 3A and 3B, and FIG. 4 which illustrates a state where the ablation is performed on the living body tissue by using the ablation device 10 in FIG. 2.

As described above, the device 10 performs the ablation on the nerve 14 internally passing through the renal artery 12 to lower the blood pressure by blocking a transmission function of the nerve 14 of a patient with intractable hypertension.

In this case, substantially similar to a procedure for delivering the general catheter 16 to the artery, a guide wire is inserted into the renal artery 12 of a living body through the femoral artery. Then, during X-ray illumination, contrast radiography is performed and the guide wire is allowed to reach the renal artery 12.

Subsequently, as illustrated in FIG. 3A, the catheter 16 left in a contracted (folded) state of the balloon 26 is inserted into the living body under the guidance of the guide wire. The user checks and ascends (delivers) the marker 34 during the X-ray illumination, and allows the distal end portion having the laser emitting unit 24 to reach a position (desired position) near the center of the renal artery 12. In the contracted state of the balloon 26, the outer diameter of the catheter 16 is sufficiently small with respect to the artery serving as a transportation route, thereby enabling the distal end portion of the catheter 16 to be smoothly delivered.

After the laser emitting unit 24 reaches a desired position, the contrast medium is supplied from the fluid supply source 52 (refer to FIG. 2). The contrast medium is supplied to the chamber 26a of the balloon 26 via the inside of the lumen 18 of the catheter 16, thereby expanding the balloon 26 inside the renal artery 12. The expanded balloon 26 contacts the inner wall surface of the renal artery 14 to thus position and fix the laser emitting unit 24 inside the renal artery 12 as shown in FIG. 3B. The positioning (expanded state of the balloon 26) causes the central axis of the catheter 16 (laser emitting unit 24) to coincide with (be centered on) the central axis of the renal artery 12.

As illustrated in FIG. 4, in the balloon 26, an expansion amount A in the radial direction of the catheter 16 is set according to the focal length D of the pulse laser PL. The expansion amount A represents a distance from the installation position of the lens 48 to the outer diameter of the balloon 26 left in the expanded state.

Here, a depth (distance X) from the intima surface 12a (blood vessel wall) of the renal artery 12 to the nerve 14 is measured each time, since there are individual differences. The nerve 14 is present on the adventitia of the blood vessel, and can be measured by measuring a thickness of the blood vessel wall. The thickness of the blood vessel wall may be measured by ultrasonic inspection. The focal length D from the lens 48 to the nerve 14 can be calculated by adding the expansion amount A when the balloon 26 is expanded to the distance X. Therefore, by appropriately setting the expansion amount A of the balloon 26, it is possible to match the focal position α with the nerve 14 (target region), and it is possible to rather easily focus the pulse laser PL.

After the positioning of the laser emitting unit 24 is completed, the pulse laser PL is emitted from the laser emitting unit 24 as illustrated in FIG. 4. In this case, the pulse laser PL is output from the laser output unit 22 (refer to FIG. 2), and the pulse laser PL is guided to the laser emitting unit 24 via the waveguide tube 20 (waveguide 44). The mirror 50 of the laser emitting unit 24 reflects the pulse laser PL toward the lens 48, and the lens 48 emits the pulse laser PL so as to be focused on the focal position α which has been set.

The pulse laser PL when emitted has a wavelength of 800 nm. Accordingly, if the pulse laser PL is emitted outward from the catheter 16 after being transmitted through the balloon 26, the pulse laser PL is transmitted through the living body tissue from the intima surface 12a of the renal artery 12 to the nerve 14. In this case, when attempting to emit the pulse laser PL to a long-distance object, the pulse laser PL is scattered inside the living body tissue, and thus it is difficult to obtain the laser intensity for performing the ablation. However, in the procedure of severing the nerve 14 of the renal artery 12, the distance X from the intima surface 12a of the renal artery 12 to the nerve 14 is sufficiently close, that is, normally approximately 2 mm or shorter. Accordingly, it is possible to guide the pulse laser PL to the focal position a while suppressing scattering.

The above-described wavelength characteristics of two-photon absorption cause the pulse laser PL transmitted to the focal position a to have the wavelength of 400 nm which is likely to be absorbed in the living body tissue. In this manner, the pulse laser PL performs the ablation on the nerve 14 and can block the transmission of the nerve 14.

In addition, the device 10 circumferentially rotates the waveguide tube 20 by using the rotary mechanism 28 (refer to FIG. 2), thereby enabling the laser emitting unit 24 to make one rotation at the positioning point of the catheter 16. In this manner, the pulse laser PL is focused on and emitted to all the circumferential directions of the renal artery 12, thereby enabling the ablation to be performed on the living body tissue (nerve 14) inside or forming a part of the renal artery.

After the completion of the ablation, the balloon 26 is contracted to release the positioning of the catheter 16, the catheter 16 is extracorporeally withdrawn (moved in the proximal direction), and the wound insertion site in the thigh is closed, thereby completing the procedure.

As described above, the device 10 according to the first embodiment emits the pulse laser PL which is guided to the laser emitting unit 24 via the waveguide 44 and focused by the lens 48, to the living body tissue. The device 10 causes the multiphoton absorption at the focal position a of the pulse laser PL. Accordingly, it is possible to perform the ablation on the nerve 14 of the renal artery 12 which corresponds to the focal position α. In this case, the pulse laser PL having the wavelength of 800 nm which is less likely to be absorbed in the living body tissue is transmitted through the living body tissue from the intima surface 12a of the renal artery 12 to the nerve 14. Then, at the focal position a, the pulse laser PL can be changed to have the wavelength of 400 nm which is likely to be absorbed in the living body tissue. The pulse laser PL having the wavelength of 800 nm is concentrated at the focal position a through the lens 48, and the pulse laser PL becomes the wavelength of 400 nm by virtue of the two-photon absorption. Accordingly, it is possible to perform the ablation on the nerve 14 of the renal artery 12 by minimizing damage to other cells of the renal artery 12 (endothelial cells which are present on the intima surface 12a). In this manner, it is possible to demonstrate the procedure in a relatively short time, without causing thrombosis or aneurysm in the renal artery 12. Therefore, it is possible to greatly reduce the burden on a patient.

In addition, since optical energy generated by emission of the pulse laser PL is used, it is possible to significantly reduce influence on a living body as compared to a case of energizing the living body.

Furthermore, the device 10 can exactly and simply set the focal length D of the pulse laser PL by using the balloon 26 in a state where the laser emitting unit 24 is positioned and fixed at the desired position inside the renal artery 12. As a result, the laser emitting unit 24 can perform the ablation by accurately focusing the pulse laser PL on the desired living body tissue.

In addition to this, the expansion of the balloon 26 allows the central axis of the catheter 16 to coincide with the central axis of the living body lumen.

Accordingly, it is possible to radially equalize the distance (focal length D) from the central axis of the catheter 16 to the living body tissue inside the renal artery 12. That is, it is possible to relatively easily center the central axis of the catheter 16 on the central axis of the renal artery 12. Therefore, without changing the focal length D of the pulse laser PL, it is possible to perform the ablation in the circumferential direction of the renal artery 12.

In this case, the balloon 26 is disposed at the position overlapped with the lens 48. Accordingly, it is possible to position and fix the laser emitting unit 24 by suppressing the positional deviation or the shaking with respect to the desired position of the renal artery 12. In addition, in a direction of the pulse laser PL emitted by the lens 48, it is possible to suppress intervention of the blood which is likely to scatter the pulse laser PL. Accordingly, it is possible to more excellently perform the ablation by using the pulse laser PL.

In addition, since the lens 48 faces the renal artery 12 from the side peripheral surface of the catheter 16, it is possible to emit the pulse laser PL from the side peripheral surface of the catheter 16. That is, when inserting the catheter 16, it is possible to easily perform the ablation on the living body lumen on the delivery route of the catheter 16.

Furthermore, the laser emitting unit 24 is rotatable in the circumferential direction of the catheter 16. Accordingly, it is possible to perform the ablation on all the nerves 14 in the circumferential direction inside the renal artery 12. Here, it has been known that the renal artery 12 has a plurality of nerves 14 which linearly extend along the axial direction (for example, refer to FIG. 2 in Japanese Laid-Open Patent Publication No. 2008-515544). In this related art, in the procedure of severing the nerve 14 of the renal artery 12, discontinuous boundary treatment is performed in which the nerve 14 in the circumferential direction of the renal artery 12 is severed by being shifted in the axial direction of the renal artery 12. Thus, the procedure requires a great deal of working hours. In contrast, the device 10 disclosed here can perform the ablation on all the nerves 14 internally passing through the renal artery 12 at the positioning point. Accordingly, it is possible to significantly improve work efficiency.

Moreover, the waveguide tube 20 having the hollow structure forms the waveguide 44. Accordingly, even the pulse laser PL having the short pulse width can be guided to the laser emitting unit 24 while the transmission losses are suppressed. Therefore, it is possible to rather easily emit the pulse laser PL having the desired laser intensity from the laser emitting unit 24.

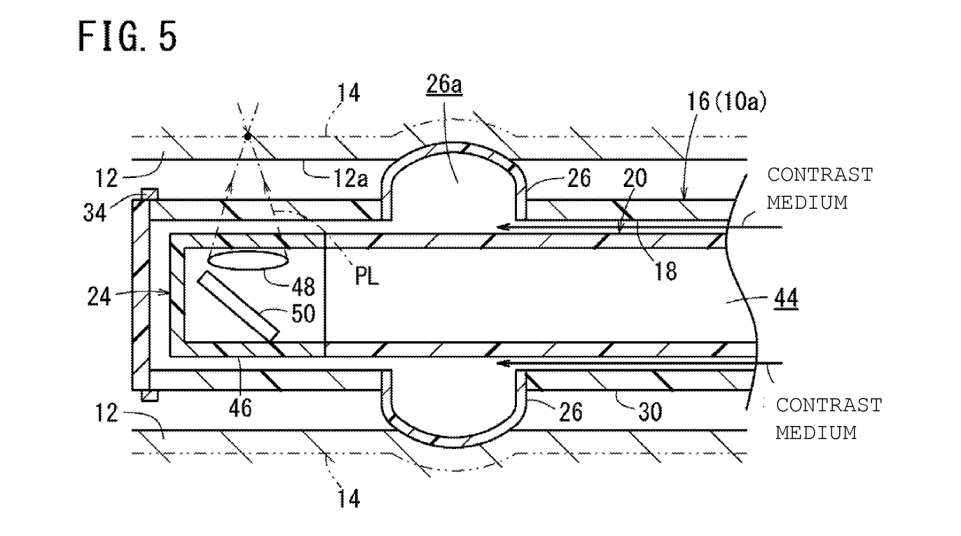

In the device 10, the balloon 26 serving as the positioning means does not need to be disposed at the position axially overlapping with the lens 48 (laser emitting unit 24) in the axial direction of the catheter 16. As a device 10a of a modification example illustrated in FIG. 5, the installation position of the balloon 26 may be shifted to a position which is not overlapped with the lens 48. In this case, it is preferable to dispose the balloon 26 at the neighboring position (axially adjacent) of the lens 48. This facilitates the positioning and the fixing of laser emitting unit 24 at the desired position in the renal artery 12.

In addition, in a state where the balloon 26 is expanded in the renal artery 12 (positioned state), it is possible to block the blood flowing in the renal artery 12. Thus, when the pulse laser PL is emitted from the laser emitting unit 24, it is possible to reduce the influence on the pulse laser PL which is caused by the flowing of the blood. Therefore, even when the balloon 26 is disposed at the position which is not overlapped with the lens 48, the laser emitting unit 24 can focus the pulse laser PL having the high intensity on the nerve 14 of the renal artery 12 and can cause the multiphoton absorption (two-photon absorption).

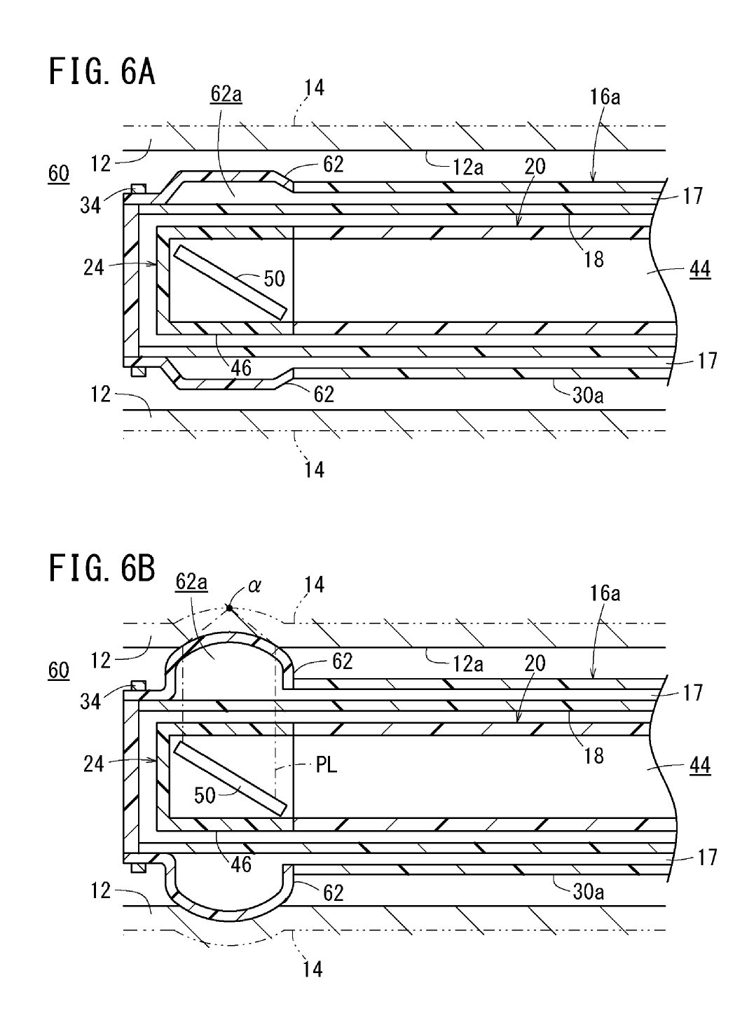

FIG. 6A illustrates an ablation device 60 according to a second embodiment representing another example of the medical device disclosed here, wherein the balloon is shown before being expanded, and FIG. 6B illustrates the medical device after the balloon has been expanded. In the device 60 according to the second embodiment, features similar to those in the medical device according to the first embodiment are identified by the same reference numerals and a detailed description of such features is not repeated.

The device 60 according to the second embodiment is different from the device 10 according to the first embodiment in that the lens is configured to include a balloon. That is, the expandable balloon according to the second embodiment has a function as, or is configured to be, a laser focusing unit which focuses the pulse laser PL at the predetermined focal position α when the balloon that includes the lens is in the expanded state. Hereinafter, the balloon of the second embodiment is referred to as a balloon lens 62.

A catheter 16a (shaft body 30a) of the device 60 has a double tube structure formed from an inner layer and an outer layer. A flow path 17 is disposed between the inner layer surrounding the lumen 18 and the outer layer forming the outer appearance layer. In the flow path 17, the fluid supply source 52 (refer to FIG. 2) is connected to the base end side, and a fluid (for example, the contrast medium) for expanding the balloon lens 62 is supplied from the fluid supply source 52.

The laser emitting unit 24, having the balloon lens 62, can be configured to have only the cap 46 and the mirror 50 without also including the lens 48. In this case, the cap 46 and the mirror 50 can be the same as the cap 46 and the mirror 50 in the first embodiment. However, the sizes of the cap 46 and the mirror 50 may be smaller by an amount that takes into account the fact that the lens 48 is not also included.

The balloon lens 62 has a circular ring (annular) shape, and similar to the balloon 26 of the first embodiment, the balloon lens 62 is disposed at an axially overlapping position with the laser emitting unit 24 (mirror 50) in the axial direction of the catheter 16a so as to circumferentially cover (surround) the laser emitting unit 24. The base end side of the balloon lens 62 is connected to the shaft body 30a, and a chamber 62a of the balloon lens 62 communicates with the flow path 17. In addition, inner and outer films of the balloon lens 62 are fixedly attached to the distal end side of the balloon lens 62 in a fluid-tight manner or in an air-tight manner.

The balloon lens 62 is in a contracted state when delivering the catheter 16a. When the catheter 16a reaches a desired position, the fluid is supplied from the fluid supply source 52 via the flow path 17 to expand the balloon lens 62 so the balloon lens 62 is in an expanded state. This enables the catheter 16a to be positioned and fixed in the renal artery 12. The balloon lens 62 (i.e., the portion which focuses the pulse laser PL at the predetermined focal position α) is thus expanded upon introduction of the fluid.

It is preferable to form the balloon lens 62 of a material exhibiting proper or desired flexibility, strength and permeability. In addition, the fluid supplied to the annular chamber 62a of the balloon lens 62 is selected from those which have an appropriate refractive index for focusing the pulse laser PL. In this case, the fluid is selected, as a matter of course, in view of a relationship with conditions (curvature, laser focusing area and the like) of a lens surface in a state where the balloon lens 62 is expanded. That is, the material forming the balloon lens 62 and the fluid supplied to the annular chamber 62a may be configured to ensure that the focal position α is located at the intended or desired position. In this manner, a laser focusing unit demonstrating the same function as that of the lens 48 according to the first embodiment is formed inside the renal artery 12.

In the balloon lens 62 left in an expanded state, the focal position α (focal length D) of the pulse laser PL is set to substantially coincide with the distance X (for example, 0.1 mm to 2 mm) from the intima surface 12a of the renal artery 12 to the nerve 14.

When performing the ablation on the nerve 14 of the renal artery 12, the pulse laser PL is emitted, from the laser emitting unit 24, toward the balloon lens 62 while the balloon lens 62 is in the expanded state. Then, when the pulse laser PL is transmitted through the balloon lens 62, the pulse laser LP is refracted and emitted according to the refractive index and the curved surface of the balloon lens 62. In this manner, the pulse laser PL can be focused on the focal position α which coincides (inclusive of substantially coincides) with the nerve 14 (target region) of the renal artery 12 so as to enable two-photon absorption to occur.

Therefore, the device 60 according to the second embodiment can obtain the same effects as those of the device 10 according to the first embodiment. Moreover, since it is not necessary to dispose the lens 48 for focusing the pulse laser LP in the laser emitting unit 24, it is possible to reduce the manufacturing cost for the ablation device 60.

The detailed description above describes an ablation device and method disclosed by way of example. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An ablation medical device to ablate living body tissue or a living body comprising:
a catheter sized to be inserted into a living body lumen of the living body;
a pulse laser output unit which outputs ultra-short pulse laser;
a waveguide in the catheter, the waveguide being connectable to the pulse laser output unit to guide the ultra-short pulse laser output by the pulse laser output unit; and
a focusing lens which focuses the ultra-short pulse laser guided along the waveguide on a focal position located on the living body tissue, and emits the pulse laser to ablate the living body tissue by causing multiphoton absorption at the focal position.

2. The ablation device according to claim 1, further comprising an inflatable balloon positioned radially outwardly of the waveguide to set the focal position of the pulse laser.

3. The ablation device according to claim 1, wherein the focusing lens is positioned inside the catheter and is rotatable relative to the catheter.

4. The ablation device according to claim 1, wherein the lens and a mirror form part of a laser emitting unit which is positioned inside the catheter.

5. An ablation device comprising:
a catheter configured to be inserted into a living body lumen;
a waveguide disposed along the catheter to guide a pulse laser output from a laser output unit; and
a laser emitting unit that includes a laser focusing unit which focuses the pulse laser guided by the waveguide on a desired living body tissue, and that emits the pulse laser to perform ablation on the living body tissue by causing multiphoton absorption at a focal position of the pulse laser.

6. The ablation device according to claim 5, further comprising positioning means for setting a focus of the pulse laser, as the laser emitting unit is positioned at a desired position inside the living body lumen.

7. The ablation device according to claim 6, wherein the positioning means is a balloon disposed on a side peripheral surface of the catheter, and which is radially outwardly expandable to an expanded state inside the living body lumen.

8. The ablation device according to claim 7, wherein the balloon in the expanded state coaxially positions the catheter in the living body lumen such that a central axis of the catheter is coaxial with a central axis of the living body lumen.

9. The ablation device according to claim 7, wherein the balloon and the laser focusing unit coaxially overlap one another.

10. The ablation device according to claim 7, wherein the balloon is located axially adjacent the laser focusing unit.

11. The ablation device according to claim 7, wherein the balloon in the expanded state is the laser focusing unit.

12. The ablation device according to claim 5, wherein the laser focusing unit is disposed to face the living body lumen from a side peripheral surface of the catheter, and wherein the laser emitting unit includes a reflection unit which guides the pulse laser guided by the waveguide to the laser focusing unit.

13. The ablation device according to claim 12, wherein the laser emitting unit is rotatable inside the catheter in a circumferential direction of the catheter.

14. The ablation device according to claim 5, wherein the laser output unit is provided in a base end side of the catheter, and wherein the laser output unit emits the pulse laser whose pulse width is shorter than that of one nanosecond.

15. The ablation device according to claim 5, wherein the waveguide is a hollow shape surrounded by a waveguide tube through which the pulse laser is transmitted.

16. A method of ablating living body tissue of a living body comprising:
positioning a waveguide in a living body lumen of the living body;
guiding a pulse laser along the waveguide; and
focusing the pulse laser guided along the waveguide at a focal position on the living body tissue and emitting the pulse laser toward the living body tissue to perform ablation on the living body tissue by causing multiphoton absorption at the focal position.

17. The method according to claim 16, wherein the living body tissue is a renal artery.

18. The method according to claim 16, wherein the pulse laser is focused by a lens positioned inside the catheter, and further comprising rotating the lens while the pulse laser is being emitted.

19. The method according to claim 16, further comprising inflating a balloon after the catheter is positioned in the living body lumen so that the balloon contacts an inner wall surface of the living body lumen to positionally fix the catheter in the living body lumen before emitting the pulse laser.

20. The method according to claim 16, wherein the pulse laser is focused by a lens forming a part of the balloon.
